(19) United States
(12) Patent Application Publication
(10) Pub. No.: US 2025/0001421 A1
(43) Pub. Date: Jan. 2, 2025

(54) TEST WELL HAVING A BOTTOM INCLUDING A SINGLE OPENING

(71) Applicant: Hewlett-Packard Development Company, L.P., Spring, TX (US)

(72) Inventors: Omer Gila, Palo Alto, CA (US); Napoleon J. Leoni, Palo Alto, CA (US); Rajesh Kelekar, Palo Alto, CA (US); Chuangyu Zhou, Palo Alto, CA (US)

(73) Assignee: Hewlett-Packard Development Company, L.P., Spring, TX (US)

(21) Appl. No.: 18/292,847

(22) PCT Filed: Aug. 5, 2021
(86) PCT No.: PCT/US2021/044770

§ 371 Date: Jan. 26, 2024

(51) Int. Cl. B01L 3/00; B01L 7/00; G01N 21/64

(52) U.S. Cl. CPC B01L 3/50851; B01L 7/52; G01N 21/6428; B01L 2200/025; B01L 2200/0647; B01L 2300/1827; B01L 2400/043; G01N 2021/6439; G01N 2201/0231

(57) ABSTRACT

A device includes at least one well to receive a polymerase chain reaction (PCR) mixture. The at least one well includes a bottom comprising an electrically resistive sheet and a carrier layer. The electrically resistive sheet is to receive a signal from a signal source to generate heat to cause a pulse-controlled amplification, thermal cycling zone in close thermal proximity to the bottom. The resistive sheet includes a first portion and a single unitary first opening aligned in a central portion of the well to enable optical detection of fluorophores from reaction of the PCR mixture in the thermal cycling zone. The transparent carrier layer is co-extensive with at least the first opening to contain the PCR mixture within the at least one well and to permit the optical detection.

### BACKGROUND

Molecular diagnostics has revolutionized modern medicine. Some types of such diagnostics may employ polymerase chain reaction (PCR) processes to rapidly make many copies of nucleic acid strands, such as RNA and/or DNA strands.

### BRIEF DESCRIPTION OF THE DRAWINGS

FIG. 1A is a diagram including a sectional side view schematically representing an example testing device including an example well to receive a polymerase chain reaction (PCR) mixture, the well including a bottom defining an opening.

FIG. 1B is side sectional view of an example electrically resistive element of a bottom of a PCR well including multiple, adjacent openings.

FIG. 2 is diagram including a side sectional view schematically representing an example testing device including an example PCR well including a bottom defining a single unitary opening and a magnetic structure adjacent to the opening.

FIG. 3 is diagram including an isometric view schematically representing an example testing device including multiple PCR wells.

FIGS. 4A and 4B each are a diagram including a sectional side view schematically representing an example testing device including an example PCR well.

FIG. 5A is a diagram including a top plan view schematically representing an electrically resistive element of a bottom of a PCR well including a first opening.

FIG. 5B is a diagram including a side sectional view schematically representing an electrically resistive element of bottom of a PCR well including a first opening relative to other components of the bottom.

FIG. 6 is a diagram including a top plan view schematically representing an electrically resistive element of a bottom of a PCR well including a first opening.

FIGS. 7, 8, 9, and 10 each are a diagram including a top plan view schematically representing an electrically resistive element of a bottom of a PCR well including a first opening and additional openings.

FIGS. 11, 12, and 13 each are a diagram including a top plan view schematically representing an electrically resistive sheet including multiple portions to form an electrically resistive element of a bottom of a PCR well including a first opening and additional openings.

FIGS. 14A and 14B each are a diagram including an isometric view schematically representing an example magnetic structure.

FIG. 15 is a diagram including a sectional view schematically representing an example testing device including a PCR well with an external magnetic structure.

FIG. 16A is a block diagram schematically representing an example operations engine.

FIGS. 16B and 16C are each a block diagram schematically representing an example control portion and an example user interface, respectively.

FIG. 17 is a flow diagram of an example method including performing a polymer chain reaction (PCR) via an example PCR well.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to an electrically resistive element for generating heat within a well for performing polymerase chain reaction (PCR) tests, such as but not limited to pulse-controlled amplification (PCA) type PCR testing. In some examples, a testing device may comprise at least one well which is to receive a polymerase chain reaction (PCR) mixture and which includes a bottom. The bottom of the at least one well may comprise an electrically resistive sheet and a carrier layer. The electrically resistive sheet is to receive a signal from a signal source to generate heat to cause a pulse-controlled amplification, thermal cycling zone in close thermal proximity to the bottom. The resistive sheet may comprise a first portion and a single unitary first opening aligned in a central portion of the well to enable optical detection of fluorophores from reaction of the PCR mixture in the thermal cycling zone. The carrier layer is co-extensive with at least the first opening to contain the PCR mixture within the at least one well and may comprise a transparent portion to permit the optical detection.

In some examples, providing a single, unitary opening within the heating element (e.g. first element or electrically resistive element) may yield a significantly enhanced power efficiency over resistive heating elements which have numerous, closely adjacent openings.

In some examples, providing a single, unitary opening within the heating element (e.g. first element or electrically resistive element) may yield a significant increase in the area of the other regions (i.e. those without an opening) of the heating element to which the PCR mixture can be exposed for thermal cycling (i.e. the thermal cycling zone). Among other aspects, this increased area may enhance repeatability and/or a limit of detection (e.g. increase sensitivity) at least because more of the PCR mixture will be exposed to the pulse-controlled amplification, thermal cycling zone.

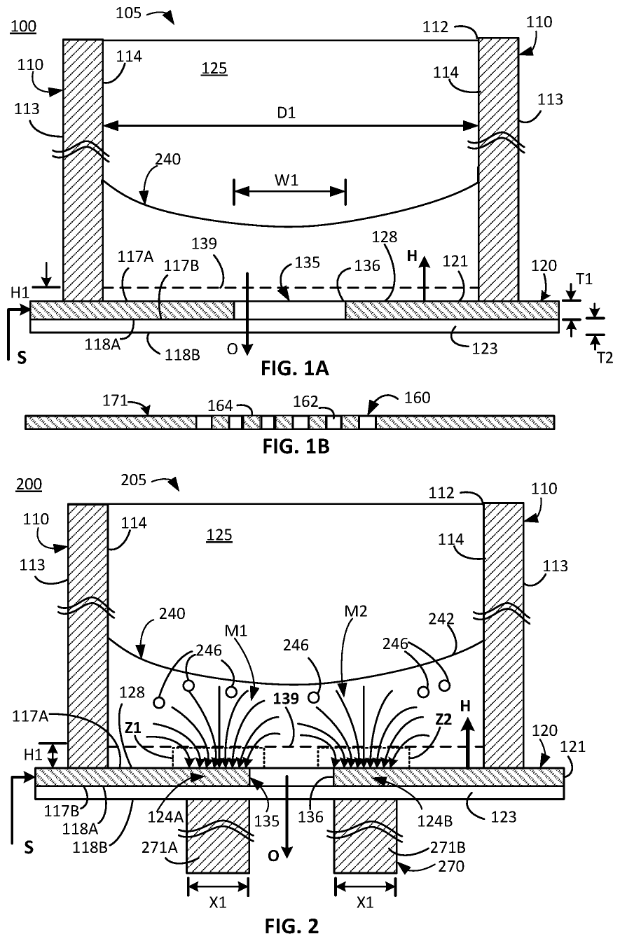

These examples, and additional examples, are described below in association with at least FIGS. 1A-17.

FIG. 1A is side sectional view of a testing device 100 comprising an example well 105 for performing a polymerase chain reaction test. As shown in FIG. 1A, the PCR well 105 comprises a bottom 120 and side wall(s) 110 extending vertically upward from the bottom 120. The bottom 120 comprises a first element 121 (e.g. layer) connected to a second element 123, such as being adhesively secured together or via other means. As further described later, the first element 121 may comprise an electrically resistive material (such as a sheet metal) suitable to generate heat within the well 105 for performing the PCR test.

The first element 121 includes a first surface 117A (e.g. internal surface) and an opposite second surface 117B (e.g. external surface), while the second element 123 (e.g. layer) includes a first surface 118A and opposite second surface 118B. In some such examples the second element 123 may comprise an inert material which includes a pressure sensitive adhesive (PSA) on its first surface 118A to facilitate securing the second element 123 to the first element 121. The second element 123 may sometimes be referred to as a carrier layer or sheet. Each side wall 110 comprises an external surface 113 and opposite internal surface 114. Together, the inner surface 114 of side walls 110 and the first surface 117A of bottom 120 define an interior 125 of the well 105, which defines a receptacle to receive a polymerase chain reaction (PCR) mixture 240. At least the inner surface 114 of side walls 110 and the first surface 117A of bottom 120 comprise, and/or are coated with, an inert material so as to not affect the PCR mixture 240 and related reaction processes.

In some examples, the side wall 110 may comprise a polymer material, such as (but not limited to) a cyclic olefin copolymer (COC) material. In some examples, the polymer material may comprise polyethylene, polypropylene, polycarbonate, polymethylmethacrylate (PMMA), and the like.

In some examples, the PCR mixture 240 comprises such PCR mixtures suitable for performing pulse-controlled amplification (PCA)-type polymerase chain reactions. Accordingly, the PCR mixture may sometimes be referred to as a PCA-PCR mixture. In some examples, overall volume of the PCR mixture 240 received into the well 105 may comprise between about 40 microliters to about 50 microliters.

In some example, the PCR mixture 240 includes components to execute three basic steps of a polymerase chain reaction via thermal cycling within the example PCR well 105. Among other components, the PCR mixture 240 may comprise beads, primers, nucleic acid strands (e.g. DNA strands, RNA strands, portions thereof), probes, and deoxyribose nucleotides (dNTPs).

A first step in thermal cycling may comprise denaturation in which the reaction volume is heated to about 94-98° C., which causes double-stranded DNA within the reaction mixture 240 to melt by breaking the hydrogen bonds between complementary bases, yielding two single-stranded DNA molecules. A second step in the thermal cycling may comprise annealing in which less heat is applied to lower the reaction temperature to about 50-65° C., which allows annealing of the primers to each of the single-stranded DNA templates as part of the reaction process. A third step of the thermal cycling may comprise extension (i.e. elongation) in which the heat applied to the reaction volume is selected to create a reaction temperature suitable for the particular DNA polymerase used. In some examples, one target activity temperature for a thermostable DNA polymerase including Taq polymerase (e.g. a thermophilic eubacterial microorganism, Thermus aquaticus) is approximately 75-80° C. In this third step, the DNA polymerase synthesizes a new DNA strand complementary to the DNA template strand by adding free nucleoside triphosphates (dNTPs) from the reaction mixture. In some examples, the temperature used in these three phases of thermal cycling may vary depending on the length of the nucleic acid strand, the time available, the type of target (e.g. RNA, DNA, etc.), the density of polymerase and primers, etc.

It will be understood that in some examples such as reverse transcriptase PCR (RT-PCR) implementations, the second and third steps (annealing and extension) may be combined and operate at a single temperature of about 65° C. In some examples, such reverse transcriptase implementations may be performed via (or as) pulse-controlled amplification (PCA) type of polymerase chain reaction.

In some examples, the thermal cycle for a polymerase chain reaction (PCR), according to a pulse-controlled amplification method, may be triggered by applying a current pulse of between about 20 Volts to about 60 Volts, and having a duration of about 0.3 to about 2 milliseconds. In some such examples, the current pulse may comprise about 40 Volts with a pulse duration of about 1 millisecond. In some such examples, the current pulse may comprise on the order of 100 amps, such as 105 amps. It will be understood that the various above-identified example values of current pulse parameters may be used to achieve a target temperature rise at the surface of about 30-40 Celsius, which may generated by a net heat flux of about 1 to about 2.5 MWatts/m\hat{ }2 applied for about 1 milliseconds. It will be understood that the above-identified parameters may vary somewhat depending on a size of the PCR well 105, volume of the PCR mixture 240, as well as the size, materials, and/or shape of the first element 121 (i.e. electrically resistive element) by which the heat is generated, etc.

In some examples, a zone in which the thermal cycling occurs may sometimes be referred to as a general thermal cycling zone (TCZ) 139 which is within a predetermined distance H1 (e.g. about 3, 4, or 5 micrometers) of the bottom 120 of the well 105 through which the heat is generated and applied. In some examples, this distance H1 may correspond to, and sometimes be referred to as, being within a close thermal proximity to the bottom. In some examples, the general thermal cycling zone also may include target thermal cycling zones where magnetic forces draw superparamagnetic beads to heighten the effectiveness of the pulse-controlled amplification of the PCR process. It will be understood that that each pulse (via the pulse-control amplification) may apply heat simultaneously in both the first and second target thermal cycling zones, in some examples.

Further details regarding such heating are described below in relation to at least the electrically resistive first element 121 of the bottom 120 of the PCR well 105.

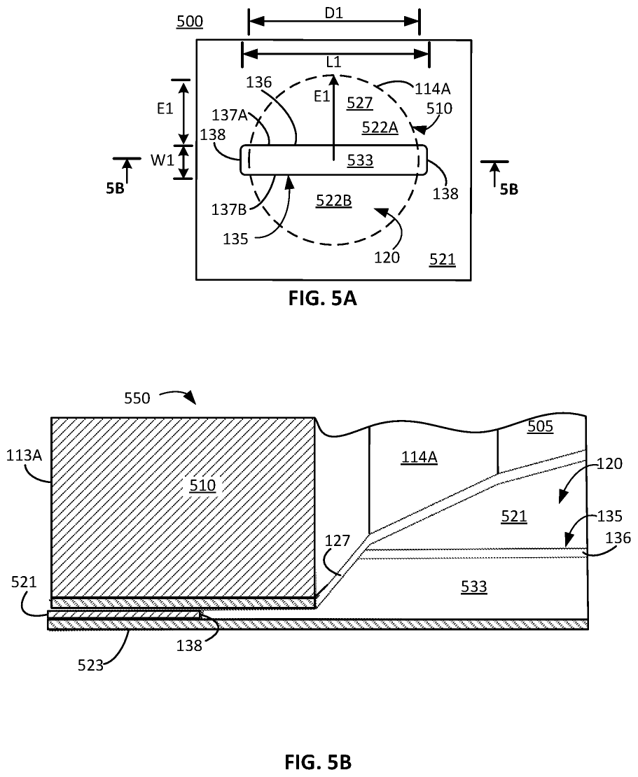

As further shown in FIG. 1A, in some examples, the electrically resistive first element 121 of bottom 120 comprises a first portion 128 and a single, unitary opening 135 with edge 136 of the first portion 128 defining the opening 135. The opening 135 may comprise a variety of shapes and sizes, and may comprise a variety of locations. In some examples, the opening 135 may comprise an elongate shape, such as a rounded rectangle (e.g. an elongate rectangular shape including rounded corners), as shown later in at least some of the examples of FIGS. 5-13.

As further shown in FIG. 1A, in some examples, the opening 135 may comprise a central location of bottom 120, at least as seen in the sectional view of FIGS. 1A-2, 4A. However, in some examples, the first opening 135 may comprise locations other than a central region of the bottom 120 of the PCR well 105.

Among other aspects, by providing a single unitary opening 135 in the electrically resistive first element 121 of bottom 120 (as compared to a heating element with numerous adjacent openings), a more robust assembly of the PCR well 105 may be achieved at least because the regions of the first element 121 used for securing relative to other components (such as second element 123) comprise relatively large uninterrupted areas which are highly amenable to adhesive processes.

As further shown in FIG. 1A, the opening 135 may comprise a width (W1) while the interior 125 of PCR well 105 may comprise a distance D1 extending between the side walls 110. Further dimensional details regarding such widths, distances, etc. are described in association with at least some later examples of the present disclosure. For instance, in some examples the PCR well 105 may comprise a generally cylindrical shape, conical shape, etc. which may be generally circular in cross-section such that distance D1 may comprise a diameter.

In some examples, the second element 123 of bottom 120 of PCR well 105 comprises a material which sealingly contains liquid within the interior 125 of PCR well 105. Accordingly, in some such examples, the second element 123 may comprise a material which is relatively impermeable to liquid, such as the components of the liquid PCR mixture. In addition, in some examples, the second element 123 comprises a transparent material though which light may be transmitted to enable optical detection (represented via directional arrow O) of output elements (e.g. fluorophores, etc.) resulting from the PCA-type, polymerase chain reaction.

In some examples, together the opening 135 in the first element 121 and the transparent material of second element 123 may comprise a window, with the edge 136 of first opening 135 defining a boundary or border of the window and the transparent second element 123 providing a liquid barrier through which light may be transmitted. It will be understood that the second element 123 is made of a material which is relatively inert relative to the components of the PCR mixture and reaction processes arising from the PCR mixture, upon heating such as via the above-identified pulse-controlled amplification, thermal cycling zone in which such reaction processes occur.

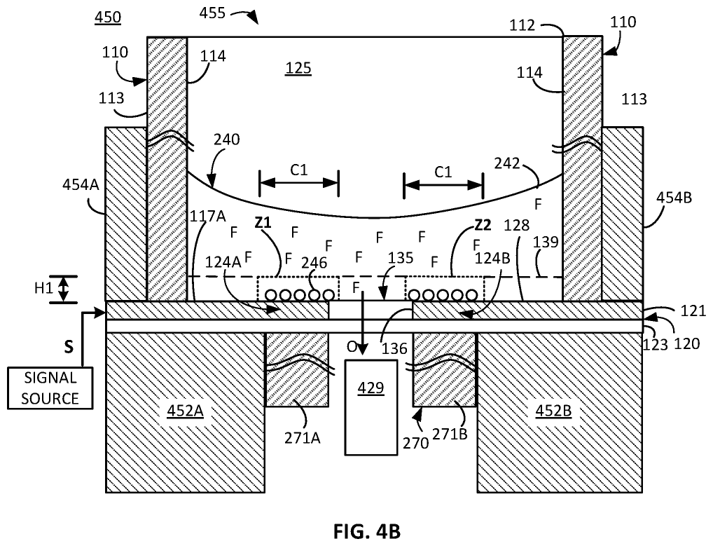

At least some example output elements of a reaction per the PCR mixture 240 may comprise fluorophores, which may be represented by reference numerals F, as later shown in at least FIG. 4A. In general terms, a fluorophore may comprise a fluorescent chemical compound that can re-emit light upon light excitation. It will be understood that output elements (e.g. labels) other than fluorophores may be optically detectable to determine a relative quantity, concentration, and/or the like of a particular analyte (e.g. virus particle, other) to which the output element is attached (e.g. bonded).

As further shown in FIG. 1A, the electrically resistive first element 121 of bottom 120 may comprise a thickness T1 between about 20 microns (e.g. micrometers) and about 50 microns, while the second element 123 of bottom 120 may comprise a thickness T2 between about 0.1 millimeter and about 1 millimeter.

With regard to these example dimensions, and other example dimensions throughout examples of the present disclosure, it will be understood that at least some components, spatial relationships, etc. in the Figures may be exaggerated (e.g. either made smaller or made larger) in scale for illustrative purposes, clarity, and/or simplicity.

As further shown in FIG. 1A, the electrically resistive first element 121 of bottom 120 may comprise a metal sheet (e.g. foil) in some examples. Moreover, the first element 121 (e.g. sheet) may be coated with a layer of gold on the order of microns (micrometers) or less than 1 micron, in some examples. Meanwhile, the second element 123 may comprise a plastic material, which may comprise polymethylmethacrylate (PMMA), polyethylene (PET), Mylar, in some examples. In some examples, the second element 123 may comprise a rubber material, such as silicone. In some examples, the entire second element 123 may be transparent as previously mentioned or may comprise a structure and/or materials which is transparent in just some regions such as a region through which optical detection is to occur, as further described later. In some examples the transparent material of element 123 may comprise minimal fluorescent properties in the wavelength detectable by the sensor.

Upon receiving a signal (S) from signal source (e.g. 433 in FIG. 4A), the first element 121 generates heat (represented via directional arrow H) for application to the PCR mixture 240 within PCR well 105. While FIG. 1A depicts a single directional arrow H, it will be understood that the heat H may be generated and applied across and along substantially the entire first surface 117A of first element 121, except at opening 135.

Moreover, via the signal source (e.g. 433 in FIG. 4A) and the electrically resistive first element 121, the heat is applied in controlled pulses in order to amplify (i.e. pulse-controlled amplification) reaction processes involving the polymerase chain reaction (PCR) mixture within a thermal cycling zone (TCZ), as represented via a dashed line 139. In some such examples, the thermal cycling zone 139 subject to a denaturation temperature (e.g. at least 90 degrees Celsius) comprises less than about 5 percent (e.g. 4.5, 4.6, 4.7, 4.8, 4.9, 5.0, 5.1, 5.2, 5.3, 5.4, 5.5) of the overall volume of the PCR mixture 240 In some examples, the thermal cycling zone 139 subject to the denaturation temperature comprises less than about 4 percent (e.g. 3.5, 3.6, 3.7, 3.8, 3.9, 4.0, 4.1, 4.2, 4.3, 4.4, 4.5) of the overall volume of the PCR mixture 240, less than about 3 percent (e.g. 2.5, 2.6, 2.7, 2.8, 2.9, 3.0, 3.1, 3.2, 3.3, 3.4, 3.5) of the overall volume of the PCR mixture 240, less than about 2 percent (e.g. 1.5, 1.6, 1.7, 1.8, 1.9, 2.0, 2.1, 2.2, 2.3, 2.4, 2.5) of the overall volume of the PCR mixture 240, or less than about 1 percent (e.g. 0.5, 0.6, 0.7, 0.8, 0.9, 1.0, 1.1, 1.2, 1.3, 1.4, 1.5) of the overall volume of the PCR mixture 240.

In some such examples, the single, unitary opening 135 in examples of the present disclosure may enhance the uniformity of the profile of heat generated from the electrically resistive first element 121, at least as compared to other designs (e.g. heating elements other than examples of the present disclosure) which may comprise a plurality of separate openings, some of which may not be centrally located. In such other designs (e.g. heating elements other than examples of the present disclosure) which have multiple openings, the heat application profile may be irregular and undesirably exhibit concentrations at edges of the multiple openings, as previously noted.

In some examples, the electrically resistive first element 121 may comprise a paramagnetic material or a ferromagnetic material.

However, in some examples, the electrically resistive first element 121 may comprise a material having a relative magnetic permeability no greater than 1.01. In some such examples, the first element 121 may sometimes be referred to as being non-magnetic at least to the extent that the material may be very weakly ferromagnetic or diamagnetic, and it is not intended to magnetically attract other objects such as beads (e.g. 246 in FIG. 4A) to the electrically resistive first element 121.

In some examples, this arrangement of the electrically first element 121 being relatively non-magnetic may enhance or contribute to a more uniform distribution of beads across the electrically resistive first element 121, which thereby reduces or avoids concentration of magnetic field lines such as might otherwise occur in some designs (other than examples of the present disclosure) where multiple separate portions of a heating element may be closely adjacent each other. The reduction in concentration of field lines via examples of the present disclosure, in turn, may reduce or avoid unwanted localization of magnetic force in such locations, which might otherwise cause the unwanted clumping or stacking of the beads. Among other aspects, these arrangements and features may lead to increased sensitivity (e.g. better limit of detection) for the PCR testing via examples of the present disclosure.

Moreover, at least because examples of the present disclosure may reduce unwanted clumping of beads, overall diffusion of other molecules (e.g. primers, DNA strands, probes, dNTPs) of the PCR mixture 240 may be increased, which contributes to overall better amplification as part of the pulse-controlled amplification of the polymerase chain reaction to occur in the thermal cycling zone 139.

In some examples, this arrangement may enable the thermal cycling zone 139 to comprise to exhibit a substantially uniform temperature, such as an area extending across the surface (e.g. 117A) of the electrically resistive first element 121 of the bottom 120 of the PCR well 105.

For instance example, by having the relative magnetic permeability of no greater than 1.01, the first element 121 of bottom 120 of the PCR well 105 may substantially prevent accumulation of the beads 246 at an edge (e.g. 136) of the first opening (e.g. 135) of the first element 121.

In some examples, in order to implement the electrically resistive first element 121 to comprise a relative magnetic permeability no greater than 1.01, a first material of the first element 121 is selected from the group of annealed stainless steel 316, brass, titanium, tantalum, tungsten, aluminum, copper, platinum, gold, silver, zinc, indium tine oxide (ITO), and combinations thereof.

In some examples, some example stainless steel materials (e.g. SS 304, 316) may be processed to make them paramagnetic or weakly ferromagnetic. In some such examples, such annealed stainless steel materials (e.g. SS 304 or SS 316) are not subjected to cold working. Alternately, at least some example austenitic steels may be heat-treated (e.g. annealed) to make the material paramagnetic or very weakly ferromagnetic at a level to meet the criteria of having a relative magnetic permeability no greater than 1.01. In some examples, one example paramagnetic aluminum material may comprise a relative magnetic permeability of 1.00002, while in some examples, one example diamagnetic copper material may comprise relative magnetic permeability of 0.99999.

Accordingly, at least some of the above-described examples may comprise materials which are diamagnetic, paramagnetic, or very weakly ferromagnetic provided that they meet the criteria of having a relative magnetic permeability no greater than about 1.01.

In some examples, achieving a relative magnetic permeability of no greater than 1.01 may be implemented via forming the first element 121 from material which omits a significant quantity of iron (Fe2), cobalt, nickel, neodymium (Nd), samarium (Sm), and the like.

With regard to at least the above-described examples in which an electrically resistive first element (e.g. 121) may comprise a material having a relative magnetic permeability of no greater than 1.01, as shown in FIG. 1B in some examples an electrically resistive first element 171 may comprise an array 160 of openings 162 in a central region of the first element 171 instead of the single opening 135 formed in the electrically resistive first element 121 in FIG. 1A. Accordingly, in some such examples, the first element 171 forms part of a PCR well comprising at least some of substantially the same features and attributes as the PCR well 105 (including first element 121) of FIG. 1A, except with the first element 171 of FIG. 1B comprising the array 160 of multiple openings 162 and the first element 171 still comprising a material having a relative magnetic permeability of no greater than 1.01. Via this arrangement, the relative magnetic permeability of the first element 171 helps to achieve the substantially uniform distribution of superparamagnetic components (e.g. beads 246 in FIG. 4A) across a surface 117A of the first element 171 instead of the PCR well 105 exhibiting unwanted concentrations of such superparamagnetic components at edges of bars 164 of the respective openings 162 in the first element 171 if the first element had a greater value of relative magnetic permeability, i.e. greater than 1.01.

It will be further understood that the examples in which the first element 121 (FIG. 1A) or first element 171 (FIG. 1B) comprises a material having a relative magnetic permeability no greater than 1.01 may be applicable to arrangements in which an electrically resistive first element of a PCR well comprises openings having a size, shape, and/or location(s) other than the single opening 135 in the first element 121 in FIG. 1A or other than the multiple, closely adjacent openings 162 in the first element 171 in FIG. 1B.

In some examples, the PCR well 105, 205 shown in FIGS. 1-4 may include a lid or cover comprising transparent materials, which may comprise materials similar to those identified herein for forming, constructing second element 123.

FIG. 2 is side sectional view schematically representing a testing device 200 including example PCR well 205. In some examples, the device 200 may comprise at least some of substantially the same features and attributes as the device 100 of FIG. 1A. As shown in FIG. 2, device 200 comprises a reaction well 205 comprising at least some of substantially the same features and attributes as reaction well 105 (FIG. 1A), except while further comprising a magnetic structure 270. As shown in FIG. 2, in some examples the magnetic structure 270 may comprise a pair of spaced apart magnetic elements 271A, 271B which are located on opposite sides of the opening 135 of the electrically resistive first element 121 of bottom 120 of well 205. In some examples, each respective magnetic element 271A, 271B may comprise a permanent magnet as further described later in association with at least FIG. 14A, while in some examples each respective magnetic element 271A, 271B may comprise a ferromagnetic element in connection with a permanent magnet, as further described later in association with at least FIG. 14B.

As further shown in FIG. 2, each magnetic element 271A, 271B is aligned with a portion 124A, 124B of the first element 121 (of bottom 120) which is immediately adjacent to the opening 135 in first element 121. Moreover, in some examples, a top portion of each magnetic element 271A, 271B may be bonded or otherwise secured relative to the second surface 118B of second element 123 of bottom 120 of PCR well 205. In some such examples, the top portion of each magnetic element 271A, 271B may comprise a size and shape (e.g. width X1) generally corresponding to a target thermal cycling zone Z1, Z2, which may comprise a region in which the beads are primarily attracted via the magnetic forces (via magnetic elements 271A, 271B) and which is within the general thermal cycling zone 139. At least some examples sizes and/or shapes of the magnetic elements 271A, 271B will be described later in association with at least FIGS. 14A, 14B.

Via this arrangement, each respective magnetic element 271A, 271B may generate magnetic field lines which produce the respective arrays of magnetic forces, as represented by the directional force arrows M1, M2. The magnetic forces draw beads 246 toward the respective portions 124A, 124B of the first element 121 of bottom 120 of PCR well 205.

Within the PCR mixture 240 (and as part of the PCA-PCR process), each respective bead 246 is functionalized with single-stranded nucleic acid such that magnetic attraction of beads 246 to the electrically resistive first element 121, such as at portions 124A, 124B, corresponds to attracting the nucleic acid strands (within the PCR mixture 240) into close thermal proximity to bottom 120 at the portions 124A, 124B above the respective magnetic elements 271A, 271B. It will be understood that the PCR mixture 240 may comprise a very high quantity of such beads 246, but few such beads 246 are shown in FIG. 2 for illustrative clarity and simplicity. In some such examples, the beads 246 may comprise a material and/or structure which is superparamagnetic with a relative magnetic permeability greater than 1.

Via the combination of the general thermal cycling zone 139 from the applied heat H and the magnetic attraction to portions 124A, 124B of bottom 120, two separate target thermal cycling zones Z1, Z2 are created in which pulse-controlled amplification (PCA) of a reaction for the PCR mixture 240 may be performed in a highly effective manner. Further details regarding example of target thermal cycling zones Z1, Z2 is further described and illustrated in association with at least FIGS. 4A-4B.

Via such arrangements, examples of the present disclosure enable testing which is more sensitive and able to detect lower quantities (or concentrations) of a particular analyte of interest (e.g. virus, other).

For instance, in one aspect relating to such examples, the overall volume of thermal cycling (to perform pulse-controlled amplification of a reaction via PCR mixture) is substantially greater than if a single thermal cycling zone were employed, which further contributes to the increased sensitivity in testing and/or ability to detect lower quantities or concentrations of particular analytes.

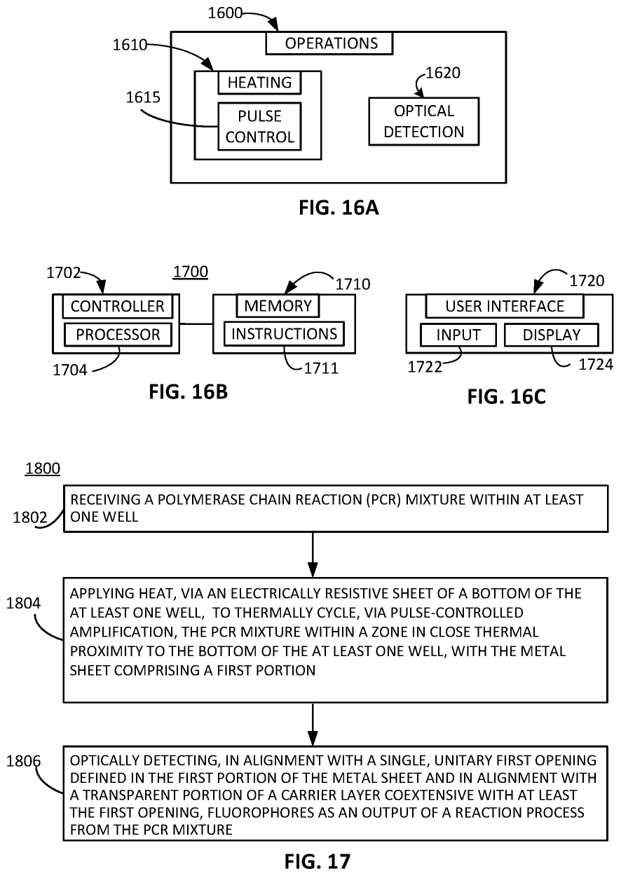

In some examples, at least some aspects of operation of, and/or monitoring of, the devices 100, 200 may be implemented via an example control portion 1700 in FIG. 16B. For instance, control portion 1700 may monitor and/or control the application of pulses from the signal source to the electrically resistive first element 121, 171, which in turn may control the generation and application of heat within the PCR well 105, 205.

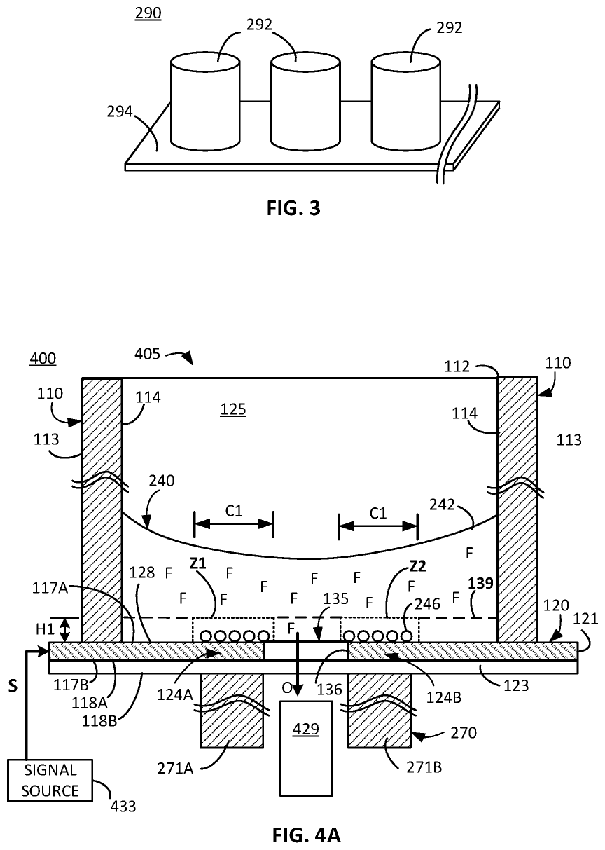

FIG. 3 is an isometric view schematically representing an example testing device 290 (e.g. molecular testing device) comprising a plurality of reaction wells 292 arranged on a common support 294. In some instances, the entire device may sometimes be referred to as a well plate or multi-well chip. In some examples, at least some of the wells 292 comprise at least some of substantially the same features and attributes including (or related to) the well 105 in FIG. 1A and/or well 205 in FIG. 2. With further reference to FIG. 3, it will be understood that testing device 290 is not limited to the number (e.g. 3) of wells 292 shown in FIG. 3, such that device 290 may comprise a greater number or lesser number of wells 292. Moreover, in some examples, testing device 290 may comprise wells 292 arranged in a two-dimensional array (e.g. 2×2, 3×2, 4×2, etc.). In some examples, the support 294 and/or individual wells 292 may comprise a portion of, and/or be in communication with, control portion (e.g. 1700 in FIG. 16B). Furthermore, in some examples the testing device 290 also may be removably connectable to a console, station, or the like to support performing, monitoring, evaluating, etc. tests in the wells 292, with the respective console (or station, other) comprising at least a portion of (or incorporating) the control portion (e.g. 1700 in FIG. 16B).

FIG. 4A is side sectional view schematically representing a testing device 400 including example reaction well 405. In some examples, the device 400 (including well 405) may comprise at least some of substantially the same features and attributes as the previously described examples device (e.g. 100, 200, etc.), except further comprising an optical detector 429 and illustrating further aspects associated with the examples of at least FIGS. 1A-3.

As shown in FIG. 4A, device 400 comprises an optical detector 429 aligned with opening 135 of the electrically resistive first element 121 of bottom 120 of the PCR well 405. In one aspect, the opening 135 is sized, shaped, and/or located relative to the optical detector 429 such that the first portion (e.g. 128 in FIGS. 1A-2, 4A-4B; 527 in FIG. 5A) of the first element 121 which generates heat does not block light transmission through the opening 135, thereby enhancing optical detection of analytes (e.g. output elements such as fluorophores F). In some examples, the optical detector 429 is to receive light indicative of a quantity or volume of certain components within the well 405. In some examples, the optical detector 429 may optically detect the presence, quantity, etc. of fluorophores (F in FIG. 4A), which are one example output element of the pulse-controlled amplified reaction per the PCR mixture 240 within well 405 (or 105, 205). In some examples, the optical detection of fluorophores F may comprise optically detecting a fluorophore signal intensity. In particular, each fluorophore F may correspond to an analyte of interest (virus particle, such as COVID 19, other) identified via the PCA-PCR reaction at least because such fluorophores are attached to components (e.g. analyte) within the PCR mixture.

As shown in FIG. 4A, by aligning the optical detector 429 with the single, unitary opening 135 (in the first element 121 of bottom 120), the effectiveness of the optical pathway (O) to detect output elements (e.g. fluorophore F) of the reaction from the PCR mixture 240 may be enhanced at least because the optical pathway intersects with the output elements (e.g. F) arising from at least two different target thermal cycling zones Z1, Z2 on opposite sides of the opening 135.

As further shown in FIG. 4A, each target thermal cycling zone Z1, Z2 may have a width as represented by C1, which may in some examples be wider than a width (X1) of each respective magnetic element 271A, 271B in FIGS. 3-4A.

As further shown in FIG. 4A, it will be understood that the electrically resistive first element 121 generates and applies heat (H) to the PCR mixture 240 within close thermal proximity to the first surface 117A of the first element 121 of bottom 120 of PCR well 405, as represented by dashed line 139, with this applied heat (H) extending across a substantially the entire diameter D1 of the PCR well 405, with exception of the generally central, single unitary opening 135 (in the first element 121 of bottom 120). As previously noted, the area denoted by dashed line 139 may sometimes be referred to as a general thermal cycling zone. It also will be understood that at least some of the heat H generated and applied by first element 121 will extend into the space above the opening 135.

It will be understood that the above-identified target thermal cycling zones (Z1, Z2) are defined in part by the general thermal cycling zone 139 and further defined in part by the portion (e.g. 124A, 124B) of the first element 121 which overlies the respective magnetic elements 271A, 271B. This arrangement, in turn, draws beads 246 into regions adjacent the opening 135, which in turn may enhance the quantity, volume, concentration, etc. of output elements (e.g. fluorophores) which would diffuse within the optical pathway via which the optical detector 429 identifies output elements (e.g. fluorophores) resulting from the PCA-PCR reactions of the PCR mixture 240 within PCR well 405.

FIG. 4B is a diagram including a side sectional view schematically representing a testing device 450 including example reaction well 455. In some examples, the device 450 may comprise at least some of substantially the same features and attributes as the device 400 of FIG. 4A, except further comprising additional heat elements external to the PCR well 455 to influence the ambient temperature around the PCR well 455 to maintain a target temperature of the overall volume of the PCR mixture 240 within the PCR well 455. In some examples, the target temperature may be between about 50 degrees C. (e.g. 49.5, 49.6, 49.7, 49.8, 49.9, 50, 50.1, 50.2, 50.3, 50.4) and about 70 degrees C. (e.g. 69.5, 69.6, 69.7, 69.8, 69.9, 70, 70.1, 70.2, 70.3, 70.4, 70.5) for desired durations, timing, etc. In some such examples, the target temperature may comprise about 65 degrees C. (e.g. 64.5, 64.6, 64.7, 64.8, 64.9, 65, 65.1, 65.2, 65.3, 65.4, 65.5).

In some examples, a pair of heat elements 452A, 452B are positioned underneath the bottom 120 of the PCR well 455 while in some examples, a pair of heat elements 454A, 454B may be positioned alongside the side walls 110 of the PCR well 455. In some such examples, the respective heat elements 452A, 452B (or 454A, 454B) may comprise a single element, such a cylindrically shaped element (e.g. ring) extending about a circumference of a cylindrically shaped PCR well 455.

Each heat element 452A, 452B, 454A, 454B may comprise a material which can generate heat, such as an electrically resistive material to generate heat upon application of a signal, or may comprise a heat-retaining material which maintains heat upon application of heat from an external source. Via such arrangements, heat generated within the PCR well 455 by the electrically resistive first element 121 of the bottom 120 of the PCR well 455 can be maintained or supplemented via the external heat elements 452A, 452B, 454A, 454B.

In some examples, just some of the heat elements 452A, 452B, 454A, 454B may be implemented to affect heat management within PCR well 455 as desired. Moreover, the heat elements 452A, 452B, 454A, 454B may comprise sizes, shapes, and/or locations other than those shown in FIG. 4B.

While not shown for illustrative simplicity, it will be understood that an additional heat element providing similar thermal functionality similar to heat elements 452A, 452B, 454A, 454B also can be placed on top of the PCR well 455 to further enhance maintaining the overall volume of the PCR mixture 240 within the PCR well 455 at a desired temperature, such as the above-noted temperature.

Among other aspects, in some examples the additional heat elements in the example of FIG. 4B may ease some performance parameters of the electrically resistive first element 121 to generate heat for PCR well 455 at least because the additional heat elements may provide heat generally to the overall volume of the PCR mixture 240 within the PCR well 455.

FIG. 5A is a diagram 500 including a top plan view schematically representing an example electrically resistive first element 521 of a bottom 520 of a PCR well (e.g. 105, 205, 405) of a testing device (e.g. 100, 200, 400). In general terms, the electrically resistive first element 521 may comprise at least some of substantially the same features and attributes as the previously described electrically resistive first element 121 and associated testing devices (e.g. 100, 200, 400) and PCR wells (e.g. 105, 205, 405).

As shown in FIG. 5A, in some examples, the first element 521 comprises an electrically resistive sheet and a single, unitary first opening 135 defined within, and by, the first element 521. The first opening 135 comprises opposite ends 138 and opposite sides 137A, 137B with edge 136 defining the first opening 135 within the first portion 527.

In some examples, the first opening 135 may comprise a rounded rectangular shape, which includes two first sides (e.g. 137A, 137B) which are spaced apart and parallel to each other with the two first sides having a first length. Meanwhile, two second sides (e.g. 138) are also spaced apart and parallel to each other with the two second sides having a second length less than the first length. Each corner of the rectangular shape is rounded, i.e. comprises an arcuate shape. In some examples, the rounded corner may comprise a radius of between about 100 micrometers and about 1 millimeter, between about 150 micrometers and 750 micrometers, between about 200 micrometers and about 500 micrometers, between about 225 micrometers and about 400 micrometers, or may comprise a radius of about 250 micrometers.

Among other features, the parallel relationship of the two first sides (e.g. 137A, 137B) of the rounded rectangular shape of opening 135 may help to maintain uniformity of current density lines while the rounded corners may lessen concentration of current that otherwise might occur if the corners were not rounded.

In some examples, the first opening 135 may comprise other shapes, such as an obround shape, an elliptical shape, and the like. In some such examples, such shapes exhibit symmetry relative to a major axis of the particular shape.

While FIG. 5A depicts just one opening, it will be understood that opening 135 is sometimes referred to as being a first opening, particularly with regard to some later described examples in association with at least FIGS. 7-13, in which a first element (e.g. like 521) may comprise additional openings (e.g. second, third, etc.) for other purposes such as heat management, current/power management, etc.

As shown in FIG. 5A, in some examples the first opening 135 comprises a length L1 which is greater than (e.g. at least as great as) a diameter D1 of an inner surface 114A (dashed lines) of side walls 110 of a PCR well (e.g. 105, 205, 405), such that the outer end portions of the first opening 135 extend laterally outside (e.g. beyond) the interior 125 of the PCR well. In this configuration, it will be understood that the second element 123 (e.g. FIGS. 1A-2, 4) of the bottom 120 is present underneath the first element 121 such that a portion 533 of the second element 123 which is exposed via first opening 135 acts to sealingly contain the PCR mixture 240 within the PCR well (e.g. 105, 205, 405).

Meanwhile, the first opening 135 comprises a width W1 (also shown in FIGS. 1A-2) which is substantially less than the diameter D1 of the PCR well between the inner surface 114A of the side walls 510. Via this arrangement, the first opening 135 is interposed between and at least partially defines the respective semicircular portions 522A, 522B on opposite sides of the first opening 135 of the bottom 120 of the PCR well 405. As further shown in FIG. 5A, each respective semicircular portion 522A, 522B comprises a radius E1. In one aspect, when considered together, the semicircular portions 522A, 522B comprise a first portion 527 within the interior of the PCR well 505. In some examples, the first portion 527 may comprise at least about 70 percent (e.g. 69.5, 69.6, 69.7, 69.8, 69.9, 70, 70.1, 70.2, 70.3, 70.4, 70.5), at least about 75 percent (e.g. 74.5, 74.6, 74.7, 74.8, 74.9, 75, 75.1, 75.2, 75.3, 75.4, 75.5), at least about 80 percent (e.g. 79.5, 79.6, 79.7, 79.8, 79.9, 80, 80.1, 80.2, 80.3, 80.4, 80.5), at least about 85 percent (e.g. 84.5, 84.6, 84.7, 84.8, 84.9, 85, 85.1, 85.2, 85.3, 85.4, 85.5), or at least about 90 percent (e.g. 89.5, 89.6, 89.7, 89.8, 89.9, 90, 90.1, 90.2, 90.3, 90.4, 90.5) of the entire area defining the bottom 120 of the interior 125 of the PCR well (e.g. 105, 205, 405).

With reference to various examples of the present disclosure which identify a first and second target thermal cycling zone (e.g. Z1, Z2 in at least FIGS. 2, 4A, 4B, 10, etc.), in some examples the portions (e.g. 124A, 124B in FIGS. 2, 4A-4B) of the electrically resistive element (e.g. 121 generally, 521 in FIG. 5A, etc.) which correspond to the target thermal cycling zones Z1, Z2 may comprise a thermal target area of at least about 20 percent (e.g. 19.5, 19.6, 19.7, 19.8, 19.9, 20, 20.1, 20.2, 20.3, 20.4, 20.5) of an entire area of the first element 121 (e.g. electrically resistive element) exposed within the PCR well (e.g. 105, 205, etc.) available for generating heat. In some such examples, this target area may be substantially greater (e.g. 2×, 3×) that pertinent heating areas in other designs (designs other than examples of the present disclosure) such that a much greater target thermal area is available for generally uniformly spreading beads (e.g. 246) (such as via magnetic attraction, in some examples) to be subject to the pulse-control amplification, thermal cycling. This arrangement, in turn, may result in establishing a monolayer or near monolayer of a higher proportion of the beads 246 (and therefore the associated single-stranded nucleic acids), which in turn significantly enhances the effectiveness of subjecting a significantly greater proportion of pertinent components of the PCR mixture 240 to the thermal cycling.

Via such arrangements, examples of the present disclosure provide a region for applying heat which is substantially greater than other designs (e.g. designs other than examples of the present disclosure) which might otherwise employ numerous, adjacent openings formed in a heating element.

It will be understood that to the extent that first portion 527 defines a certain percentage (e.g. 70 percent) of the entire area defining the bottom 120 of the interior 125 of the PCR well (e.g. 105, 205, 405), the first opening 135 would define a complementary percentage (e.g. 30 percent) of the entire area defining the bottom 120 of the interior 125 of the PCR well (e.g. 105, 205, 405).

FIG. 5B is a diagram including a partial side sectional view of an testing device 550 including an example PCR well 505 that incorporates the first element 521, as taken along lines 5B-5B in FIG. 5A. In some examples, the testing device 550 (including PCR well 505) comprises at least some of substantially the same features and attributes as the previously described example devices (and PCR wells) in association with at least FIGS. 1A-5A.

With reference to FIG. 5A and as further shown in FIG. 5B, the bottom 120 of the PCR well 505 comprises electrically resistive first element 521 including the single unitary opening 135 defined by edge 136, which exposes transparent portion 533 of second element 523 (like second element 123 in FIGS. 1A-4B). As further shown in FIG. 5B, end 138 of opening 135 is located external to the inner surface 114A of the side wall 510 of the PCR well 505. In some examples, the PCR well 505 also comprises an adhesive layer 127 to facilitate securing the vertical portions (e.g. side walls 510) of the PCR well 505 relative to the bottom 120, such as the first element 521.

With reference to at least both FIGS. 5A-5B, in some examples, the first portion 527 of the bottom 120 from (and through) which heat is applied into the PCR mixture 240 (to provide a thermal cycling zone) may comprise at least 2×, 3×, or 4× the area of heating (e.g. in which a pulse-controlled amplification zone may be located) as compared to at least some other designs (e.g. designs other than examples of the present disclosure) which may involve numerous adjacent openings at the bottom of a well.

In some such examples, the example first element 521 may yield a much higher power efficiency in terms a significantly higher percentage of overall applied power being available for use within an interior 125 of the PCR well, as compared to at least some designs which may involve numerous adjacent openings at the bottom of a PCR well.

Moreover, via deployment of the single, unitary first opening 135 (versus multiple, separate openings) having a generally uniform shape (e.g. rounded rectangle without sharp corners), power is distributed substantially uniformly along an entire length of the first opening 135. In some such examples, this highly uniform power distribution corresponds to the power exhibiting a standard deviation of less than 5 percent along a length of the opening 135. In some examples, the standard deviation may comprise less than 4 percent, or less than 3 percent. In some such examples, this substantially uniform power distribution also may enable a general thermal cycling zone (e.g. 139) and/or target thermal cycling zones (e.g. Z1, Z2) which is substantially uniform in terms of the temperatures produced such that the thermal cycling zone may be understood as being unified or a single thermal cycling zone of a particular temperature range. This arrangement stands in contrast to some other designs (e.g. designs other than examples of the present disclosure) in which a lack of uniformity of power at, around, near numerous openings in the bottom of a PCR well may produce different or incongruent thermal cycling zones arising from different regions of the bottom of the PCR well producing different temperature profiles. At least in this sense, such different thermal cycling zones (based on different temperature profiles) arise unintentionally at least because of lack of uniformity in power applied in different regions of the bottom of the PCR well.

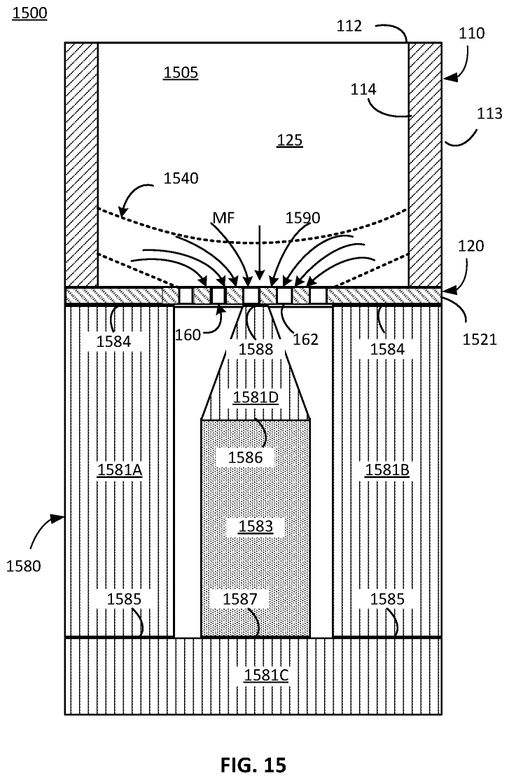

It will be understood that some example PCR wells including a first element 521 including a single, unitary opening 135, such as shown in FIGS. 5A-5B (or other FIGS.), may be implemented in some example PCR well without a magnetic structure, such as the magnetic structure 271 in FIGS. 2, 4A and/or the magnetic structures of FIGS. 14A-15. In such examples, the components of the PCR mixture 240 to be heated via the first element 521 may become subject to the thermal cycling zone upon gravitational forces bringing such components within the thermal cycling zone.

Moreover, in some examples, as previously noted, in some examples the first element 521 may be formed of a material which is relatively non-magnetic, such as having a relative magnetic permeability no greater than 1.01, such that components of the PCR mixture including magnetic features, such as superparamagnetic beads (e.g. 246 in FIG. 4A) will become substantially uniformly distributed across the bottom 120 of the PCR well, and particularly substantially uniformly distributed across the first element 521 which generates heat for the thermal cycling zone (e.g. 255 in FIG. 4A).

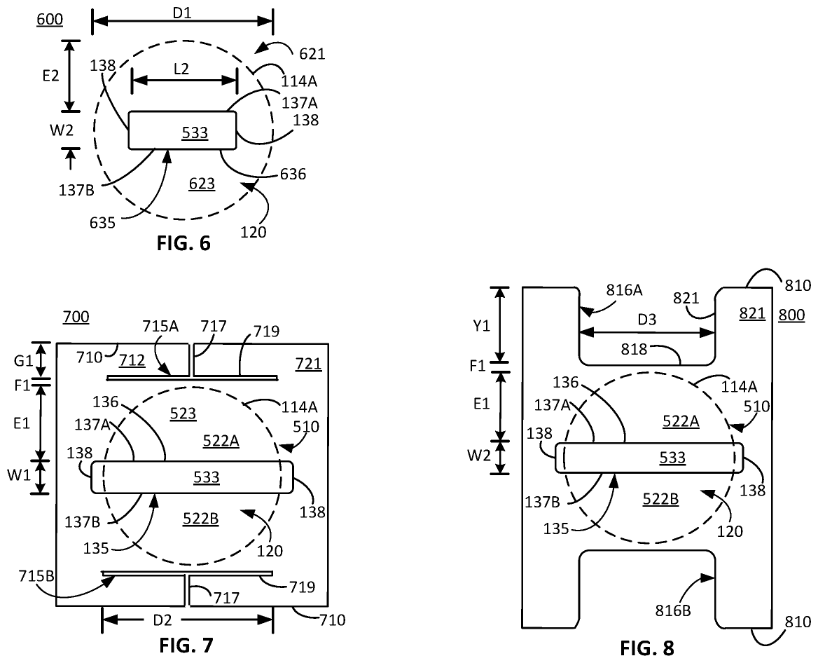

FIG. 6 is a diagram 600 including a top plan view schematically representing an example electrically resistive first element 621 of a bottom 120 of a PCR well (e.g. 105, 205, 405) of a testing device (e.g. 100, 200, 400). In some examples, the electrically resistive first element 602 comprises at least some of substantially the same features and attributes as the example first element 521 in FIGS. 5A-5B, except with a first opening 635 in first element 621 comprising a width W2 which is different from (e.g. greater than) the width W1 of first opening 135 in FIG. 5A and comprising a length L2 which is different from (e.g. less than) the length L1 of the first opening 135 in FIG. 5. Accordingly, as shown in FIG. 6, in some examples the length L2 of first opening 635 is less than a diameter D1 of the interior 125 of the PCR well within the interior surface 114A of the PCR well.

In some examples, while the first portion 623 of the first element 621 in FIG. 6 defines a first opening 635 having a width and a length different from the width and length of the first opening 135 in FIG. 5, it will be understood that the first portion 623 in FIG. 6 may comprise the same percentages (e.g. at least about 70 percent, at least about 75 percent, at least about 80 percent, at least about 85 percent, or at least about 90 percent) of the entire area defining the bottom 120 of the interior 125 of the PCR well (e.g. 105, 205, 405), as was described for first portion 523 in association with FIG. 5.

FIG. 7 is a diagram 700 including a top plan view schematically representing an example electrically resistive first element 721 of a bottom 120 of a PCR well (e.g. 105, 205, 405) of a testing device (e.g. 100, 200, 400). In some examples, the example first element 721 comprises at least some of substantially the same features and attributes as the example first element 521 in FIG. 5, except with the first element 721 further comprising second and third openings 715A, 715B on opposite sides of the first opening 135, as shown in FIG. 7. In particular, in some examples each respective second and third openings 715A, 715B define a slit, which may have a T-shape in some examples. For instance, each second and third opening 715A, 715B may comprise a base 717 and transverse member 719 which together define a slit (or slit-type opening) starting at edge 710 of the first element 721. In some such examples, the transverse member 719 may extend on both sides of the base 717 and may have a length substantially similar to the diameter D1 of the interior 125 of the PCR well (e.g. diameter D1 of the bottom 120 of the PCR well).

In some examples, the second and third openings 715A, 715B may extend a distance G1 from the edge 710 toward the first opening 135. Moreover, as shown in FIG. 7, in some examples each respective second and third opening 715A, 715B are located external to the interior surface 114A of the side walls 510 of the PCR well. In some examples, the respective second and third openings 715A, 715B are spaced apart from each other, being located on opposite sides of the first opening 135 and on opposite sides of the PCR well (e.g. 105, 205, 405) defined by side wall 510.

In some examples, the transverse member 719 of each respective second and third opening 715A, 715B extends generally parallel to a longitudinal axis (i.e. length) of the first opening 135. As shown in FIG. 7, in some examples, a width F1 of the transverse member 719 (and of base 717) of each respective second and third openings 715A, 715B may be substantially less than a width W1 of the first opening 135. In some such examples, in this context “substantially less” may comprise about at least about 30 percent less, at least about 35 percent less, at least about 40 percent less, and so on.

In some examples, the first opening 135 may sometimes be referred to as an optical opening, at least to the extent that the first opening 135 may be provided for optically detecting output elements (e.g. fluorophores) arising from the pulse-controlled amplification, polymerase chain reaction from mixture 240, as previously described in relation to at least FIGS. 1A, 2, and 4A. However, it will be understood the shape and/or size of the first opening 135 also is selected to achieve substantially uniformity in current density and related electrical parameters, which in turn may enhance uniformity in thermal properties and uniformity in distribution of beads (which have been functionalized with single-stranded nucleic acids of the PCR mixture), and the like. Accordingly, to the extent that the term optical opening may sometimes be used, the term optical is not to be understood as limiting the features of the opening 135 to being solely optically-related.

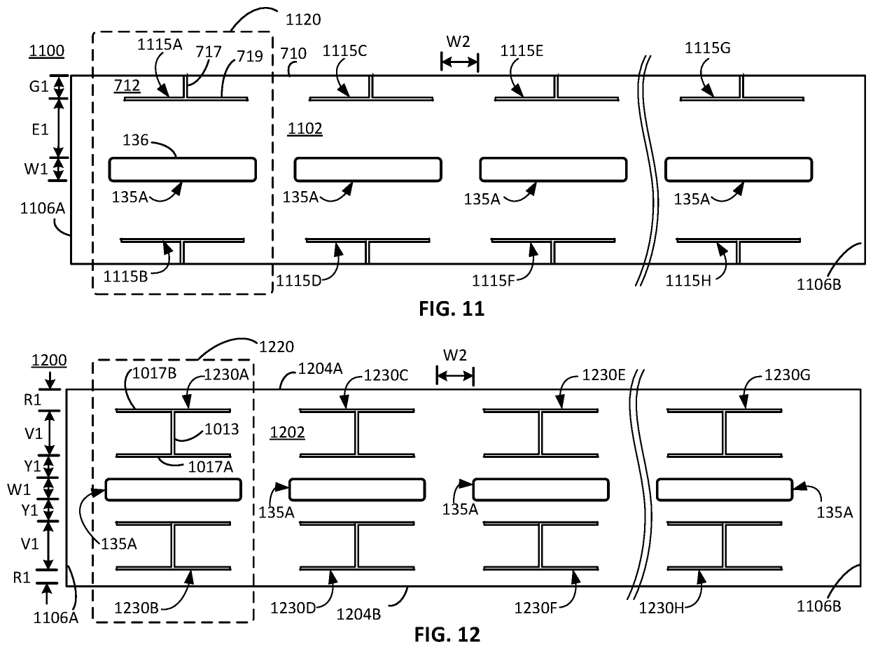

Meanwhile, in some examples, the respective second and third openings 715A, 715B may sometimes be referred to as resistivity-reduction openings, at least to the extent that the second and third openings 715A, 715B (i.e. slits) are provided, at least, to reduce the resistivity between adjacent wells on a well plate, such as further described in association with at least FIG. 11. Via this arrangement, more power is made available to the first portion 523 of the bottom 120 within the interior 125 of the PCR well.

In some examples, instead of the generally T-shaped openings 715A, 715B (e.g. slits) of first element 721, other shaped, slit-type openings such as the H-shaped, slit-type openings may be employed. It will be understood that other shaped and/or sized openings may be employed instead of the previously described T-shaped or H-shaped openings.

In some examples, the slit-type openings in FIG. 7 (T-shaped or H-shaped) generally comprise slit portions having a linear or straight edges facing each other. However, in some examples, the slit-type openings may comprise slit portions having a zigzagged shape.

At least some aspects of the example arrangement of FIG. 7 are further described later in association with at least FIG. 11.

FIG. 8 is a diagram 800 including a top plan view schematically representing an example electrically resistive first element 821 of a bottom 120 of a PCR well (e.g. 105, 205, 405) of a testing device (e.g. 100, 200, 400). In some examples, the electrically resistive first element 821 comprises at least some of substantially the same features and attributes as the electrically resistive first element 721 in FIG. 7, except with the first element 821 comprising second and third openings 816A, 816B (on opposite sides of the first opening 135, as shown in FIG. 8) taking the form of generally rectangular shaped cut-outs. In particular, the first element 821 may be formed without rectangular-shaped portions corresponding to the size and shape of openings 816A, 816B (e.g. cut-outs) or may be formed by cutting-out (e.g. cutting away) corresponding shaped and sized rectangular portions from a whole sheet (from which first element 821 is formed) to achieve openings 816A, 816B.

In some examples, the second and third openings 816A, 816B may extend a distance Y1 (e.g. depth) from the edge 810 of first element 821 toward the first opening 135 (but terminate prior to the first opening 135). Moreover, as shown in FIG. 8, in some examples each respective second and third opening 816A, 816B is located external to at least the interior surface 114A of the side walls 510 of the PCR well and, in some examples may be located completely external to side walls 510. In some examples, the respective second and third openings 816A, 816B are spaced apart from each other, being located on opposite sides of the first opening 135 and on opposite sides of the PCR well (e.g. 105, 205, 405). In some examples each respective second and third opening 816A, 816B define a length D3 substantially similar to the diameter D1 of the interior 125 of the PCR well (e.g. diameter D1 of the bottom 120 of the PCR well).

Via this arrangement, the current applied through the electrically resistive first element 821 within the PCR well remains generally unaffected but more power becomes available to the portion of the first element 821 within the PCR well due to the absence of the material of first element 821 in the area of the openings 816A, 816B.

It will be further understood that examples, such as the example first element 721 of FIG. 7 having slit-type openings 715A, 715B may be implemented instead of the example first element 821 having cut-out type openings 816A, 816B to achieve similar results regarding reducing resistivity between adjacent wells (e.g. wells on a well plate) but without removing as much material used to form the portions of first element 821 external to the PCR well.

At least some aspects of the example arrangement of FIG. 8 are further described later in association with at least FIG. 12.

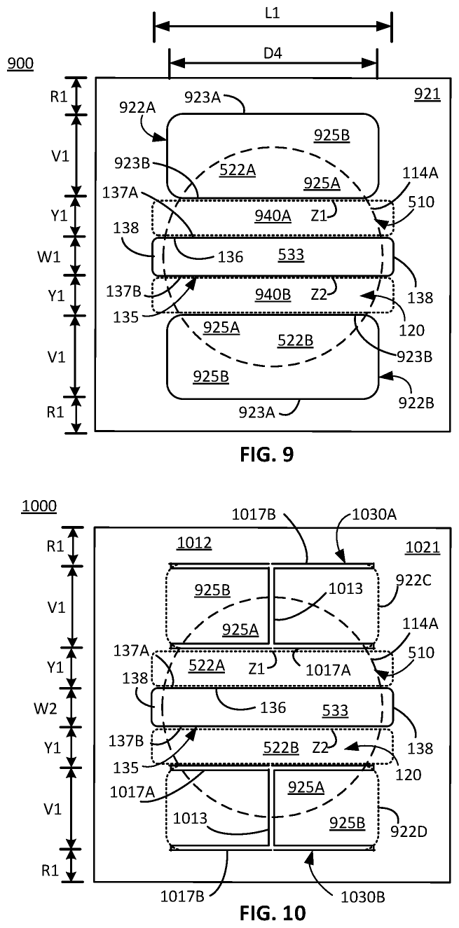

FIG. 9 is a diagram 900 including a top plan view schematically representing an example electrically resistive first element 921 of a bottom 120 of a PCR well (e.g. 105, 205, 405) of a testing device (e.g. 100, 200, 400). In some examples, the example first element 921 comprises at least some of substantially the same features and attributes as the first element 821 in FIG. 8, except with the electrically resistive first element 921 comprising second and third openings 922A, 922B (on opposite sides of the first opening 135, as shown in FIG. 9). In some examples, as shown in FIG. 9, the second and third openings 922A 922B may comprise a rounded rectangular shape. In some examples, the second and third openings 922A, 922B may comprise a shape substantially similar to a shape (e.g. rounded rectangle) of the first opening 135 but may have a different size (e.g. different width, different length).

In some examples, the second and third openings 922A, 922B may be positioned at an interior location spaced apart by a distance R1 from outer edge 910 of the first element 921. In some examples, the second and third openings may comprise a width V1, which is greater than a width W1 of the first opening. In some examples, the respective second and third openings 922A, 922B are spaced apart from each other, being located on opposite sides of the first opening 135 and on opposite sides of the PCR well (e.g. 105, 205, 405). In some examples, each respective second and third opening 922A, 922B define a length D4 substantially similar to the diameter D1 of the interior 125 of the PCR well (e.g. diameter D1 of the bottom 120 of the PCR well).

In contrast to the examples of FIGS. 7-8, as shown in FIG. 9, in some examples a portion 925A of each respective second and third opening 922A, 922B is juxtaposed with, and overlaps a portion 522A of the bottom 120 of the interior 125 within the PCR well, i.e. interior of the inner surface 114A of the side walls 510. As further shown in FIG. 9, a first side 923B of each respective second and third opening 922A, 922B is spaced apart from the respective sides 137A, 137B of the first opening 135 by a distance Y1.

In some examples, this distance Y1 may correspond to the width X1 of each respective magnetic element 271A, 271B shown in FIGS. 2 and 4A. Accordingly, the portion 940A (e.g. 124A in FIG. 2, 4) of the first element 921 between the second opening 922A and side edge 137A of the first opening 135 and the portion 940B (e.g. 124B in FIGS. 2, 4A) of the first element 921 between the third opening 922B and side edge 137B of the first opening 135 generally correspond to the position and orientation of the respective magnetic elements 271A, 271B in the example of FIG. 2. Moreover, in some examples, the respective portions 940A, 940B also correspond to the target thermal cycling zones Z1, Z2 (FIGS. 2, 4A, 4B). In some such examples, the respective magnetic elements 271A, 271B may work to attract magnetic beads (e.g. 246 in FIG. 2) into the respective target thermal cycling zones Z1, Z2, which may enhance the effectiveness of the thermal cycling zones by bringing more components of the desired reaction processes to be subject to the pulse-controlled amplification process caused by heating in close thermal proximity to the bottom 120 of the PCR well.

In some examples, the magnetic elements 271A, 271B (FIGS. 2, 4A) are omitted and instead, a magnetic structure having a size, shape, and/or location other than that shown for elements 271A, 271B (FIGS. 2, 4A) may be implemented to draw beads 246 (which are functionalized with single-stranded nucleic acids of the PCR mixture) toward and into the target thermal cycling zones Z1, Z2.

However, it will be understood that in some examples, such magnetic elements (e.g. 271A, 271B in FIG. 2) are omitted and components of PCR mixture may migrate relative to bottom 120 according to gravitational forces. In some such examples, the target thermal cycling zones Z1, Z2 will still be present to perform pulse-controlled amplification.

Meanwhile, the remaining portion 925B of each respective second and third opening 922A, 922B is located external the bottom 120 of the interior 125 of the PCR well 905, such as being external to at least the inner surface 114A of the side walls 510 (or external to the entire side wall 510, such as side wall 110 in FIGS. 1A-2).

Consistent with the examples of at least FIGS. 1A-2 and 4, the openings 922A, 922B in electrically resistive first element 921 do not extend through the second element 123 (underneath the first element 921) such that second element 123 continues to sealingly contain the contents (e.g. PCR mixture) within the interior 125 of the PCR well. Moreover, in some examples in the region of the second and third openings 922A, 922B, the second element 123 comprises an opaque material and/or an opaque cover which overlies the transparent material of the second element 123 in those regions. Via this arrangement, ambient light and/or other undesired light intrusion into the PCR well is prevented so as to protect the integrity, effectiveness, and/or accuracy of the optical detection of optical output elements (e.g. fluorophores) within the PCR well as described in various examples of the present disclosure.

Instead of the second element 123 comprising a transparent material with the exception of the region of the second and third openings 922A, 922B (of the first element 121 of the bottom 120 of the PCR well), in some examples the second element 123 may comprise a generally opaque material except with the second element 123 comprising a transparent material in the region (e.g. 533 in FIGS. 5B-10) of the first opening 135 of the first element 121 through which the optical detection is to be performed.

Via the arrangement of the second and third openings 922A, 922B in the electrically resistive first element 921, the absence of the electrically resistive material in portions 925A, 925B of the first element 921 effectively makes such portions 925A, 925B into non-heating regions such that portions of a PCR mixture 240 (within the PCR well) located above the portions 925A, 925B are not directly heated within the PCR well, which may help to maintain a temperature of the overall volume of the PCR mixture 240 within the PCR well.

In some examples, the absence of resistive material in portions 925A, 925B of the first element 921 also may help to increase power applied for heating in the portions 940A, 940B of the first element 921 at the target thermal cycling zones Z1, Z2. Via such arrangement, the increased power at the target thermal cycling zones Z1, Z2 may enhance the pulse-controlled amplification in the target thermal cycling zones Z2, Z2.

FIG. 10 is a diagram 1000 including a top plan view schematically representing an example electrically resistive first element 1021 of a bottom 120 of a PCR well (e.g. 105, 205, 405) of a testing device (e.g. 100, 200, 400). In some examples, the electrically resistive first element 1021 comprises at least some of substantially the same features and attributes as the first element 921 in FIG. 9, except with the electrically resistive first element 1021 comprising second and third openings 1030A, 1030B (on opposite sides of the first opening 135, as shown in FIG. 10). In some examples, as shown in FIG. 10, each respective second and third opening 1030A, 1030B may comprise a slit-type opening. In some such examples, the second and third openings 1030A, 1030B may comprise a shape substantially similar to a shape of the T-shaped slit-style openings 715A, 715B in FIG. 7, except for the openings 1030A, 1030B in FIG. 10 comprising an H-shaped slit-style opening.

As shown in FIG. 10, each H-shaped opening 1030A, 1030B may comprise a slit having a pair of spaced apart, slit portions 1017A, 1017B which are generally parallel to each other and a centrally located slit portion 1013 extending transversely between, and connected to, the elongate slit portions 1017A, 1017B. The slit portions 1017A, 1017B extend generally parallel to a longitudinal axis of the first opening 135, with a length of such slit portion 1017A, 1017B having a length substantially the same as a diameter (e.g. D4 in FIG. 9) extending across the interior 125 (FIG. 2, 4) of the bottom 120 of the PCR well.

As further shown in FIG. 10, in some examples the H-shaped slit-style openings 1030A, 1030B of first element 1021 may define a periphery, as represented via dashed lines 922C, 922D, which generally corresponds to the generally rectangular shape of the openings 922A, 922B of FIG. 9. In addition to this general shape relationship, in some examples the H-shaped slit-style openings 1030A, 1030B of first element 1021 may provide substantially the same or similar electrical properties regarding resistivity, desired current density lines, power distribution, heat profile, etc. as the full rectangular shaped openings 922A, 922B in FIG. 9, except with the H-shaped slit-style openings 1030A, 1030B preserving more material of the first element 121. Accordingly, like the rounded rectangular openings 922A, 922B of FIG. 9, the H-style openings 1030A, 1030B may minimize or prevent heating of the overall volume of the PCR mixture 240 within the PCR well, while helping to increase a temperature of the PCR mixture 240 in the thermal cycling zone (e.g. Z1, Z2).

Via such arrangements, this material preservation may enhance operating performance of the first element 1021 while still achieving the electrical performance characteristics like those of the openings 922A, 922B in the example of FIG. 9. In some examples relating to FIG. 10, the slit which defines openings 1030A, 1030B may be filled with a filler to prevent exposure of a pressure sensitive adhesive on the second element 123 to the components of the PCR mixture 240 within the PCR well. In some examples, the filler may comprise a sheet metal filler which is not electrically resistive like the electrically resistive material generally defining the first element 1021.

Via the arrangement of openings 1030A, 1030B in first element 1021, power is distributed substantially uniformly in the target thermal cycling zones Z1, Z2. In some such examples, this highly uniform power distribution corresponds to the power exhibiting a standard deviation of less than about 2 percent (e.g. 1.8, 1.9, 2, 2.1, 2.2) along a length of the opening 135. In some examples, the standard deviation may comprise less than 1 percent (e.g. 0.8, 0.9, 1, 1.1, 1.2) or less than about 0.5 percent (e.g. 0.4, 0.45, 0.5, 0.55, 0.60). In some such examples, this substantially uniform power distribution also may enable each separate thermal cycling zone Z1, Z2 to be substantially uniform in terms of the temperatures produced. Accordingly, each thermal cycling zone Z1, Z2 may be understood as being unified or as a single thermal cycling zone of a particular temperature range.

In contrast, some designs (other than examples of the present disclosure) which lack of uniformity of power at, around, near numerous adjacent openings in the bottom of a PCR well may produce different thermal cycling zones arising from different regions of the bottom of the PCR well producing different temperature profiles. At least in this sense, such different thermal cycling zones (based on different temperature profiles) arise unintentionally at least because of lack of uniformity in power applied in different regions of the bottom of the PCR well. Accordingly, such designs (other than examples of the present disclosure) fail to define separate multiple (e.g. two) thermal cycling zones, each of which independently maintains a substantially uniform temperature profile across a respective thermal cycling zone.

Among other features, the arrangement of the second and third openings 1030A, 1030B of the first element 121 may provide a substantial increase (e.g. 2×, 3×) in the area of the target thermal cycling zone (e.g. Z1, Z2) as compared to some designs (other than examples of the present disclosure), which therefore may result in substantial increase in the effectiveness of pulse-controlled amplification (PCA).

FIG. 11 is diagram 1100 including a top plan view schematically representing an example electrically resistive sheet 1102 to act as a first element of a bottom of a corresponding array of to-be-constructed PCR wells. In some examples, the sheet 1102 may be used to form a well plate or well chip including a series of PCR wells in a manner similar to the example depicted in FIG. 3. As shown in FIG. 11, sheet 1102 is formed to include a series of spaced apart first openings 135A (e.g. opening 135 in FIGS. 1A-10) and pairs of second and third openings (e.g. 1115A, 1115B; 1115C, 1115D; 1115E, 1115F; and 1115G, 1115H). As represented via dashed line 1120, one set of a first opening 135A, second opening 1115A, and third opening 1115B correspond to an arrangement to provide for an example PCR well, such as those represented in at least FIGS. 1A-2 and 4-7. As shown in FIG. 11, the respective first openings 135A (and an associated pair of respective second and third openings (e.g. 1115A, 11151B)) are spaced apart from each other along a length of sheet 1102 to provide enough space for the formation of separate PCR wells (represented by dashed lines 1120) using the electrically resistive sheet 1102. In some examples, the electrically resistive sheet 1102 (e.g. a metal foil in some examples) may comprise at least some of substantially the same features and attributes as the first element (e.g. 121 in FIGS. 1A-2, 4-7, etc.) as previously described in association with at least FIGS. 1A-10.

FIG. 12 is diagram 1200 including a top plan view schematically representing an example electrically resistive sheet 1202 to act as a first element of a bottom of a corresponding array of to-be-constructed PCR wells. In some examples, the sheet 1202 may be used to form a well plate or well chip including a series of PCR wells in a manner to the example depicted in FIG. 3. As shown in FIG. 12, sheet 1202 is formed to include a series of spaced apart first openings 135A (e.g. opening 135 in FIGS. 1A-10) and pairs of second and third openings (e.g. 1230A, 1230B; 1230C, 1230D; 1230E, 1230F; and 1230G, 1230H). As represented via dashed line 1220, one set of a first opening 135A, second opening 1230A, and third opening 1230B correspond to an arrangement to provide an example PCR well, such as those represented in FIGS. 1A-2, 4-6, and 9-10. As shown in FIG. 12, the respective first openings 135A (and an associated pair of respective second and third openings (e.g. 1230A, 1230B)) are spaced apart from each other along a length of sheet 1202 to provide enough space for the formation of adjacent, separate PCR wells (as represented by dashed lines 1220) using the electrically resistive sheet 1202. In some examples, the electrically resistive sheet (e.g. a metal foil in some examples) may comprise at least some of substantially the same features and attributes as the first element (e.g. 121 in at least FIGS. 1A-2, 4-6, and 9-10 etc.) as previously described in association with at least FIGS. 1A-10.

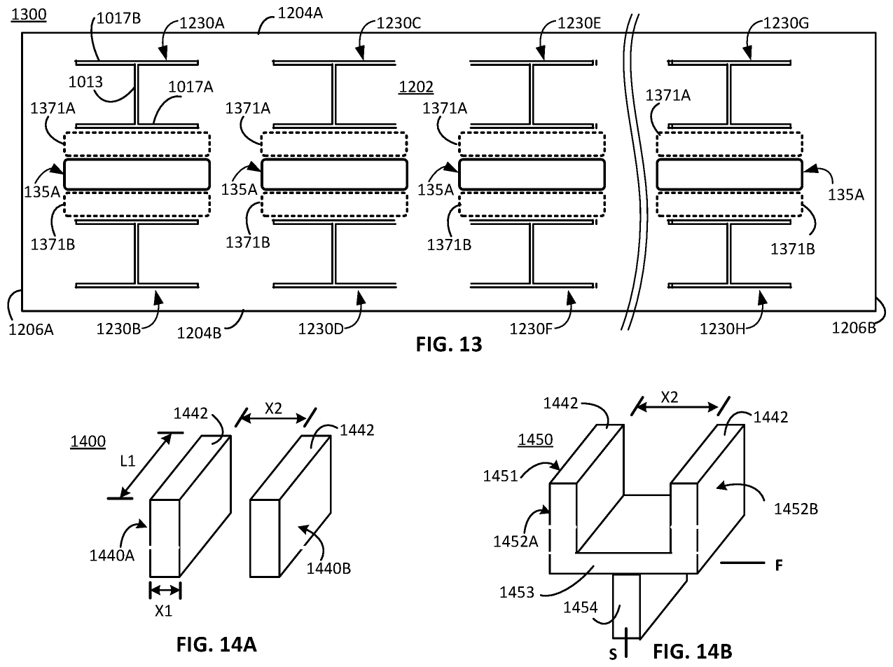

FIG. 13 is a diagram 1300 including a top plan view schematically representing example electrically resistive sheet 1302 like sheet 1202 in FIG. 12, except further depicting a location of magnetic elements 1371A, 1371B (shown in dashed lines) generally corresponding to the example arrangements in FIGS. 2 and 4 which include magnetic elements 271A, 271B. In some examples, the dashed lines representing magnetic elements 1371A, 1371B (like 271A, 271B in FIGS. 2, 4A) also may represent a first and second target thermal cycling zones Z1, Z2 as in the examples of FIGS. 2, 4A, 9 and 10.

FIG. 14A is a diagram including an isometric view schematically representing a magnetic structure 1400 such as a pair of magnetic elements 1440A, 1440B. In some examples, the magnetic elements 1440A may comprise at least some of substantially the same features and attributes as, and/or provide one example implementation of, the magnetic elements 271A, 271B in FIGS. 2, 4A and/or 1371A, 1371B in FIG. 13. Moreover, in some such examples, the magnetic elements 1440A, 1440A may comprise permanent magnets. In some examples, the permanent magnets may comprise Neodymium Iron Boron (NeFeB) alloy magnets, which may be coated in Nickel in some examples. However, it will be understood that other permanent magnet materials may be used in some examples.

In some examples, each magnetic element 1440A, 1440B may comprise a generally rectangular bar shape having a length (L1), and width (X1) like those dimensions shown in FIGS. 2, 4A. When deployed as part of a testing device (e.g. 100, 200, 400) including a PCR well (e.g. 105, 205, 405), in some examples the magnetic elements 1440A, 1440B may be spaced apart from each other by a distance X2 which corresponds to a width of a first opening, such as a width (W1) of a first opening 135 in a first element (e.g. 121 in FIGS. 1A-2, 4A) of a bottom 120 of a PCR well (e.g. 105, 205, 405). Via such spacing, the respective magnetic elements 1440A, 1440B are positioned to be located on opposite sides of a central opening, such as opening 135 in at least FIGS. 2, 4A, etc. However, the distance X2 may comprise other values as may be suited to differently configured openings of an electrically resistive first element of a bottom of a PCR well.

In some examples, a top portion 1442 of each respective magnetic element 1440A, 1440B is sized and/or shaped to establish connection with a second element 123 of a bottom 120 of a PCR well, in a manner similar shown for magnetic elements 271A, 271B in FIGS. 2 and 4A being aligned with portions 214A, 214B of first element 121 in FIG. 2.

FIG. 14B is a diagram including an isometric view schematically representing an example magnetic structure 1450. In some examples, the magnetic structure 1450 may comprise at least some of substantially the same features and attributes as, and/or include components which provide one example implementation of, the magnetic elements 271A, 271B in FIGS. 2, 4A and/or 1371A, 1371B in FIG. 13.

As shown in FIG. 14B, in some examples the magnetic structure 1450 comprises a U-shaped ferromagnetic element 1451 supported by a permanent magnet 1454. As shown in FIG. 14B, in some examples the U-shaped ferromagnetic element 1451 may comprise a base 1453 which extends in a first orientation, with the base 1453 supported by and connected to the permanent magnet 1454. In some examples, the permanent magnet 1454 may extend in a second orientation (S) perpendicular to the first orientation (F). Meanwhile, the U-shaped ferromagnetic element 1451 comprises a pair of arms 1452A, 1452B spaced apart from each other and extending generally parallel to each other, with the respective arms 1452A, 1452B extending in the second orientation, transverse to the base 1453 of the U-shaped ferromagnetic element 1451. In a manner similar to that described for FIG. 14A, each arm 1452A, 1452B comprises a top portion 1442 for establishing contact with, and connecting to, a second element (e.g. 123 in FIGS. 2, 4A) of a bottom (e.g. 120 in FIGS. 2, 4A) of a PCR well, in a manner similar to that depicted in at least FIGS. 2, 4A.

FIG. 15 is a diagram including a sectional view schematically representing an example testing device 1500 including a PCR well 1505 with an external magnetic structure 1580. In some examples, the testing device 1500 may comprise at least some of substantially the same features as the examples in association with at least FIGS. 1B (and/or other examples), while explicitly including an example external magnetic structure 1580. Like the electrically resistive first element 171 in FIG. 1AB, the electrically resistive first element 1521 in FIG. 15 may comprise an array 160 of openings 162 to enable optical detection of fluorophores (or other output elements) resulting from reaction processes in the PCR mixture 1540 within the PCR well 1505. As further shown in FIG. 15, in some examples the magnetic structure 1580 may comprise a centrally-located, vertically-extending permanent magnet 1583 and several ferromagnetic elements 1581A, 1581B, 1581C, 1581D. For example, the ferromagnetic element 1581D may comprise a generally trapezoidal shape or other shapes and is connected to a first end 1586 of the permanent magnet 1583 while a tip 1588 of the ferromagnetic element 1581D is connected to an available surface of the bottom 120 of the PCR well 1505. Meanwhile, an opposite second end 1587 of the permanent magnet 1583 is connected to transversely-extending ferromagnetic element 1581C, which may act as an overall base for the magnetic structure 1580. As further shown in FIG. 15, a pair of vertically-extending ferromagnetic elements 1581A, 1581B are generally parallel to each other, and spaced apart from each other. A first end 1584 of respective elements 1581A, 1581B is connected to an available surface of the bottom 120 of the PCR well 1505, while an opposite second end 1585 of the respective elements 1581A, 1581B is connected to the base 1581C.

Via this arrangement of magnetic structure 1580, magnetic fields are produced at bottom 120 of the PCR well 1505 which yields magnetic forces 1590 as represented by the directional arrows MF illustrating magnetic attraction, such as to magnetically attract beads (e.g. 246 in FIG. 4A) within a PCR mixture 1540 (like PCR mixture 240 in at least FIGS. 2, 4A, etc.). As shown in FIG. 15, the magnetic forces MF are aligned to draw beads (e.g. 246 in FIGS. 2, 4A) toward and into a thermal cycling zone juxtaposed with the array 160 of openings 162 and bars 164 (FIG. 1AB) at which heating via the electrically resistive material of first element 1521 occurs.

In a manner similar to that described in at least some examples of the present disclosure, magnetic attraction of the beads in this manner may help facilitate a more effective reaction processes of the PCR mixture during thermal cycling because more beads (and therefore more nucleic acid strands) would be present before or during the reaction process.

FIG. 16A is a block diagram schematically representing an example operations engine 1600. In some examples, the operations engine 1600 may form part of a control portion 1700, as later described in association with at least FIG. 16B, such as but not limited to comprising at least part of the instructions 1711. In some examples, the operations engine 1600 may be used to implement at least some of the various example devices and/or example methods of the present disclosure as previously described in association with FIGS. 1A-15 and/or as later described in association with FIGS. 16B-17. In some examples, the operations engine 1600 (FIG. 16A) and/or control portion 1700 (FIG. 16B) may form part of, and/or be in communication with, a testing device (including at least one polymerase chain reaction (PCR) well) such as the example devices and methods described in association with at least FIGS. 1A-15 and 16B-17.

In some examples and in general terms, the operations engine 1600 directs, monitors, and/or reports information regarding a polymerase chain reaction (PCR) to occur within at least one well of a testing device, with the polymerase chain reaction (PCR) comprising a pulse-controlled amplification (PCA) type of polymerase chain reaction in some examples. As shown in FIG. 16A, in some examples the operations engine 1600 may comprise a heating engine 1610 and an optical detection engine 1620. The heating engine 1610 may track and/or control heating within a PCR well, such as via applying pulses of an electric signal from signal source to an electrically resistive element (e.g. metal foil) forming at least a portion of a bottom of the PCR well, as described in association with at least FIGS. 1A-2 and 4A-4B, etc. In some such examples, the heating engine 1610 may track and/or control the heating according to a pulse-controlled amplification (PCA) parameter 1615 to perform the polymerase chain reaction (PCR) within the PCR well (e.g. 105, 205) via pulse-controlled amplification.

In some examples, the optical detection engine 1620 may track and/or control optical detection of aspects of a polymerase chain reaction within a PCR well (e.g. 105, 205), such as but not limited to, optical detection of fluorophores (or other output elements) as an output of the polymerase chain reaction processes. In some such examples, a volume or quantity of the detected fluorophores may be indicative of a presence, intensity, prevalence, etc. of a particular analyte (e.g. viral particle, other) within the sample associated with the reaction mixture deposited within the well (e.g. 105, 205). In some examples, the optical detection engine 1620 implements the optical detection via optical detector (e.g. 429 in FIG. 4A).

It will be understood that various engines and parameters of operations engine 1600 may be operated interdependently and/or in coordination with each other, in at least some examples.

FIG. 16B is a block diagram schematically representing an example control portion 1700. In some examples, control portion 1700 provides one example implementation of a control portion forming a part of, implementing, and/or generally managing the example testing devices (e.g. molecular testing devices), as well as the particular portions, components, PCR wells, signal sources, electrically resistive elements, heat elements, magnets, optical detectors, operations, control portion, instructions, engines, functions, parameters, and/or methods, as described throughout examples of the present disclosure in association with FIGS. 1A-16A and 16C-17. In some examples, control portion 1700 includes a controller 1702 and a memory 1710. In general terms, controller 1702 of control portion 1700 comprises at least one processor 1704 and associated memories. The controller 1702 is electrically couplable to, and in communication with, memory 1710 to generate control signals to direct operation of at least some of the example molecular testing devices, as well as the particular portions, components, wells, signal sources, electrically resistive elements, heat elements, magnets, optical detectors, operations, control portion, instructions, engines, functions, parameters, and/or methods, as described throughout examples of the present disclosure. In some examples, these generated control signals include, but are not limited to, employing instructions 1711 stored in memory 1710 to at least direct and manage testing operations via examples of the present disclosure. In some instances, the controller 702 or control portion 1700 may sometimes be referred to as being programmed to perform the above-identified actions, functions, etc.

In response to or based upon commands received via a user interface (e.g. user interface 1720 in FIG. 16C) and/or via machine readable instructions, controller 1702 generates control signals as described above in accordance with at least some of the examples of the present disclosure. In some examples, controller 1702 is embodied in a general purpose computing device while in some examples, controller 1702 is incorporated into or associated with at least some of the example molecular testing devices, as well as the particular portions, components, PCR wells, signal sources, electrically resistive elements, heat elements, magnets, optical detectors, operations, control portion, instructions, engines, functions, parameters, and/or methods, etc. as described throughout examples of the present disclosure.

For purposes of this application, in reference to the controller 1702, the term “processor” shall mean a presently developed or future developed processor (or processing resources) that executes machine readable instructions contained in a memory or that includes circuitry to perform computations. In some examples, execution of the machine readable instructions, such as those provided via memory 1710 of control portion 1700 cause the processor to perform the above-identified actions, such as operating controller 1702 to implement testing operations via the various example implementations as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 1710. The machine readable instructions may include a sequence of instructions, a processor-executable machine learning model, or the like. In some examples, memory 1710 comprises a computer readable tangible medium providing non-volatile storage of the machine readable instructions executable by a process of controller 1702. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 1702 may be embodied as part of at least one application-specific integrated circuit (ASIC), at least one field-programmable gate array (FPGA), and/or the like. In at least some examples, the controller 1702 is not limited to any specific combination of hardware circuitry and machine readable instructions, nor limited to any particular source for the machine readable instructions executed by the controller 1702.

In some examples, control portion 1700 may be entirely implemented within or by a stand-alone device.

In some examples, the control portion 1700 may be partially implemented in one of the example testing devices and partially implemented in a computing resource separate from, and independent of, the example devices but in communication with the example testing devices. For instance, in some examples control portion 1700 may be implemented via a server accessible via the cloud and/or other network pathways. In some examples, the control portion 1700 may be distributed or apportioned among multiple devices or resources such as among a server, a testing device, a user interface.

In some examples, control portion 1700 includes, and/or is in communication with, a user interface 1720 as shown in FIG. 16C. In some examples, user interface 1720 comprises a user interface or other display that provides for the simultaneous display, activation, and/or operation of at least some of the example testing devices, as well as the particular portions, components, PCR wells, signal sources, electrically resistive elements, heat elements, magnets, optical detectors, operations, control portion, instructions, engines, functions, parameters, and/or methods, etc., as described in association with FIGS. 1A-16B and 17. In some examples, at least some portions or aspects of the user interface 1720 are provided via a graphical user interface (GUI), and may comprise a display 1724 and input 1722.

FIG. 17 is a flow diagram of an example method 1800. In some examples, method 1800 may be performed via at least some of the testing devices, as well as the particular portions, components, PCR wells, signal sources, electrically resistive elements, heat elements, magnets, optical detectors, operations, control portions, engines, functions, parameters, and/or methods, etc. as previously described in association with at least FIGS. 1AA-7C. In some examples, method 800 may be performed via at least some testing devices, as well as the particular portions, components, PCR wells, signal sources, electrically resistive elements, heat elements, magnets, optical detectors, coating, molding, operations, control portions, engines, functions, parameters, and/or methods, etc. other than those previously described in association with at least FIGS. 1A-16C.

As shown at 1802 in FIG. 17, in some examples method 1800 comprises receiving a polymerase chain reaction (PCR) mixture within at least one well. As further shown at 1804 in FIG. 17, in some examples method 1800 comprises applying heat, via an electrically resistive sheet of a bottom of the at least one well, to thermally cycle, via pulse-controlled amplification, the PCR mixture within a zone in close thermal proximity to the bottom of the at least one well, with the electrically resistive sheet comprising a first portion. As further shown at 1806 in FIG. 17, in some examples method 1800 comprises optically detecting, in alignment with a single, unitary first opening defined in the first portion of the resistive sheet and in alignment with a transparent portion of a carrier layer coextensive with at least the first opening, fluorophores as an output of a reaction process from the PCR mixture.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A device comprising:
at least one well to receive a polymerase chain reaction (PCR) mixture and including a bottom comprising:
an electrically resistive sheet to receive a signal from a signal source to generate heat to cause a pulse-controlled amplification, thermal cycling zone in close thermal proximity to the bottom, wherein the resistive sheet comprises a first portion and a single unitary first opening aligned in a central portion of the well to enable optical detection of fluorophores from reaction of the PCR mixture in the thermal cycling zone; and
a transparent carrier layer co-extensive with at least the first opening to contain the PCR mixture within the at least one well and to permit the optical detection.

2. The device of claim 1, wherein the first opening comprises a rounded rectangular shape.

3. The device of claim 1, wherein the first opening has a length greater than a diameter of the bottom of the PCR well.

4. The device of claim 1, wherein the first portion of the electrically resistive sheet comprises at least about 70 percent of an entire area of the bottom of the at least one well.

5. The device of claim 1, wherein the electrically resistive sheet includes a second opening and a third opening located on opposite sides of, and spaced apart from, the first opening.

6. The device of claim 5, wherein the second and third openings are located laterally external to side walls at least partially defining the bottom of the at least one well.

7. The device of claim 5, wherein at least a portion of the second and third openings at least partially define the bottom of the well, and at least a portion of the carrier layer is co-extensive with the respective second and third openings with the carrier layer being opaque at least in a region corresponding to the respective second and third openings.

8. The device of claim 1, wherein the PCR mixture comprises beads functionalized with single-stranded nucleic acids of the PCR mixture and wherein the beads comprise superparamagnetic material with a relative magnetic permeability greater than 1, and the device comprising:
at least one external magnet aligned to apply a magnetic force to the beads with the single-stranded nucleic acids, into the thermal cycling zone, within the at least one well to cause the beads with the nucleic acid strands to travel into, and remain present within, the pulse-controlled amplification, thermal cycling zone.

9. A device comprising:
at least one well to receive a polymerase chain reaction (PCR) mixture and including a bottom comprising:
an electrically resistive sheet, which is to receive a signal from a signal source to cause the resistive sheet to generate heat to form a pulse-controlled amplification, thermal cycling zone within the at least one well in close thermal proximity to the bottom, wherein the resistive sheet comprises a single, unitary first opening aligned in a central portion of the well; and
a carrier layer co-extensive with at least the first opening of the resistive sheet to contain the reaction mixture within the at least one well and including a transparent portion; and
an optical detector aligned with the first opening in the resistive sheet to optically detect, through the transparent portion of the carrier layer, a fluorophore signal intensity within the at least one well as an output of a reaction process from the PCR mixture.

10. The device of claim 9, wherein the first opening comprises a rounded rectangular shape, which has a length greater than a diameter of the bottom of the at least one well.

11. The device of claim 9, wherein the resistive sheet comprises a first portion which defines the first opening, wherein the first portion comprises at least about 70 percent of an entire area of the bottom of the at least one well,
wherein the resistive sheet comprises a second opening and a third opening located on opposite sides of, and spaced apart from, the first opening, and
wherein the resistive sheet comprises at least one of:
the second and third openings located laterally external to the bottom of the well; and
at least a portion of the second and third openings at least partially define the bottom of the well, and at least a portion of the transparent carrier layer is co-extensive with the respective second and third openings.

12. A method comprising:
receiving a polymerase chain reaction (PCR) mixture within at least one well;
applying heat, via an electrically resistive metal sheet of a bottom of the at least one well, to thermally cycle, via pulse-controlled amplification, the PCR mixture within a zone in close thermal proximity to the bottom of the at least one well, with the metal sheet comprising a first portion;
optically detecting, in alignment with a single, unitary first opening defined in the first portion of the metal sheet and in alignment with a transparent portion of a carrier layer coextensive with at least the first opening, fluorophores as an output of a reaction process from the PCR mixture.

13. The method of claim 12, wherein the PCR mixture comprises beads, which are functionalized with single-stranded nucleic acids of the PCR mixture, and which are superparamagnetic with a relative magnetic permeability greater than 1, and the method comprises:
applying an external magnetic force to the superparamagnetic beads within the at least one well to cause the superparamagnetic beads with the single-stranded nucleic acids to travel into, and remain present within, the pulse-controlled amplification, thermal cycling zone.

14. The method of claim 13, wherein the application of heat comprises the first portion of the metal sheet comprising at least about 70 percent of an entire area of the bottom of the at least one well.

15. The method of claim 13, wherein first opening comprises a rounded rectangular shape and wherein the application of heat comprises the resistive sheet including (at least) a second opening and a third opening located on opposite sides of, and spaced apart from, the first opening, and at least one of:
the second and third openings located laterally external to the bottom of the well; and
at least a portion of the second and third openings at least partially define the bottom of the well, and at least a portion of the carrier layer is co-extensive with the respective second and third openings.
